(19) United States
(12) Patent Application Publication
(10) Pub. No.: US 2020/0317926 A1
(43) Pub. Date: Oct. 8, 2020

(54) NANOPARTICLE DISPERSIONS

(71) Applicant: Sensient Colors UK Ltd., Hertfordshire (GB)

(72) Inventor: Paul Wallace, Royston (GB)

(21) Appl. No.: 16/763,312

(22) PCT Filed: Nov. 26, 2018
(86) PCT No.: PCT/GB2018/053411

§ 371 Date: May 12, 2020

(30) Foreign Application Priority Data
Nov. 27, 2017 (GB) 1719693.2

(51) Int. Cl. C09B 67/46; A61K 8/04; C09D 11/322; B01F 5/04

(52) U.S. Cl. CPC C09B 67/0086; A61K 8/04; C09B 67/0089; B01F 2005/0034; C09D 11/322; B01F 5/0405; C09B 67/009

(57) ABSTRACT

A method for preparing a dispersion of nanoparticles of a solid organic dye or pigment in a liquid carrier, the method comprising forming a solution or slurry of the solid organic dye or pigment in an organic or other solvent, and continuously mixing the solution or slurry with the liquid carrier in a counter current or concurrent mixing reactor providing a dispersion of the nanoparticles in the liquid carrier and solvent mixture and, optionally concentrating the dispersion.

The present invention is concerned with a method for preparing nanoparticle dispersions of solid organic dyes or pigments in a liquid carrier, such as an aqueous based liquid carrier, and with nanoparticle dispersions of solid organic dyes or pigments obtained by the method. The nanoparticle dispersions may be used as ink concentrates for digital inkjet printing or as cosmetic pastes.

The production of ink concentrates suitable for digital inkjet printing presently requires milling of solid pigments or dyes to a suitable nanoparticle size and subsequent dispersion of the nanoparticles in a suitable carrier liquid together with stabilisers, such as wetting agent and/or dispersant.

The production process is a batch process which is often time consuming and costly not least because the required milling is generally protracted and consumes a great deal of energy and large amounts of water and organic solvent.

Furthermore, it is often accompanied by significant problems in reproducibility of the dispersion and the inkjet ink and is limited by the fact that certain pigments or dyes (for example, Disperse Red 55) cannot easily be milled by standard processes to provide stable dispersions.

The present invention generally seeks to improve upon this situation by providing a method for the direct production (viz. without the need for milling) of nanoparticle dispersions of solid organic dyes or pigments.

The present invention also seeks to provide a method for the continuous production of nanoparticle dispersions of solid organic dyes or pigments.

Accordingly, in a first aspect, the present invention provides a method for preparing a dispersion of nanoparticles of a solid organic dye or pigment in a liquid carrier, the method comprising forming a solution or slurry of the organic dye or pigment in an organic or other solvent and continuously mixing the solution or slurry with the liquid carrier in a counter current or concurrent mixing reactor to obtain a dispersion of the nanoparticles in the liquid carrier and solvent mixture.

The method may further comprise concentrating the dispersion by removal of the organic or other solvent and, optionally, removal of a portion of the liquid carrier.

Note that references herein to dispersions of nanoparticles of a solid organic dye or pigment are references to dispersions of nanoparticles largely comprising the solid organic dye or pigment and having an average particle size below 500 nm.

The nanoparticles may consist essentially of the solid organic dye or pigment in the liquid carrier. Alternatively, the nanoparticles may consist essentially of the solid dye or pigment and a wetting agent encapsulating, at least in part, the nanoparticles.

References to a solid organic dye or pigment are references to a synthetic or naturally occurring organic dye or organic pigment which comprises an organic or organometallic molecule and is generally a solid at standard temperature and pressure.

The solid organic dye or pigment may be a crystalline solid, a colloidal solid (such as a quantum dot) or an amorphous solid.

The method is not limited to any particular class of organic dye or pigment—it being sufficient that the solid organic dye or pigment has some solubility in the organic or other solvent at a suitable temperature and pressure.

In some embodiments, the molecular weight of the solid organic dye or pigment is less than 1500 g/mol, for example, less than 1200 g/mol, 1000 g/mol or 900 g/mol.

Note further that references to a liquid carrier are references to a liquid in which the solid organic dye or pigment is generally insoluble at standard room temperature and pressure.

The organic or other solvent and the liquid carrier may, therefore, be considered as respectively a solvent and an anti-solvent for the solid organic dye or pigment. The liquid carrier of the solvent mixture will normally be present in amount in excess of the organic or other solvent.

Of course, the organic or other solvent and the liquid carrier should be miscible with each other.

Suitable counter current and concurrent mixing reactors include those described in the literature as continuous hydrothermal flow synthesis (CHFS) reactors and used for the synthesis of metals or metal oxides.

The counter current mixing reactors generally comprise an inlet for a first solution, an inlet for a second solution and an outlet for both the first and second solution.

Although the counter current mixing reactor may comprise a T-shaped or Y-shaped reactor, it is preferred that it comprise a reactor in which the second inlet is diametrically opposed to the first inlet and is disposed in the outlet.

Preferred counter current mixing reactors are described in International Patent Applications WO 2005/077505 A2, WO 2014/111703 A2 and WO 2015/075439 A1 (all of which are incorporated in their entirety by reference herein).

The counter current mixing reactor may, therefore, have a vertical configuration in which the first inlet, the second inlet and the outlet are co-axially disposed. The second inlet may comprise a shaped nozzle, in particular, a conical funnel.

The reactor may also be provided with a preheater for heating one of the solution and liquid carrier and a cooler for cooling the other of the solution and liquid carrier.

Note that there is no chemical reaction in the reactor but only an intimate mixing of the solution or slurry and the liquid carrier which results in the precipitation or formation of nanoparticles of the organic compound in the liquid carrier and solvent mixture.

In a preferred embodiment, the solution or slurry is fed upwards through the first inlet and the liquid carrier fed downwards through the second inlet. Alternatively, the liquid carrier may be fed upwards through the first inlet and the solution or slurry fed downwards through the second inlet.

The method may comprise forming a solution or slurry of the solid organic compound containing a wetting agent and/or dispersant. Alternatively, or additionally, the method may comprise mixing the solution or slurry with a liquid carrier containing a wetting agent and/or a dispersant.

The inclusion of a wetting agent in the organic or other solvent and/or the liquid carrier may provide for encapsulation of nanoparticles of the solid organic dye or pigment as soon as they are formed in the counter current mixing reactor.

The addition of a dispersant to the liquid carrier and solvent mixture may facilitate the encapsulation of the nanoparticles of the solid organic dye or pigment—and may be carried out prior to, or after, removing the organic or other solvent from the mixture.

In one embodiment, the liquid carrier contains only a wetting agent and the method further comprises adding a dispersant to the nanoparticle dispersion obtained at the outlet prior to, or after, the removal of the organic or other solvent.

The method surprisingly provides for nanoparticle dispersions of solid organic dyes and pigments which are stable (even without the inclusion or addition of wetting agent and/or dispersant) and well-suited to the production of ink concentrates for inkjet printing.

First, the dispersions are unimodal and show fairly narrow nanoparticle size distribution around a central peak and a mean diameter between 1 nm and 500 nm, in particular, between 100 nm and 300 nm, and for example, around 120 nm.

Secondly, the dispersions may show median Dᵥ50 values between 100 nm and 300 nm and, in particular, around 120 nm. The dispersions may alternatively show Dᵥ97 values between 100 nm and 300 nm and, in particular around 120 nm.

In preferred embodiments, the method comprises forming a solution of the solid organic dye or pigment in an organic solvent. In these and other embodiments, the liquid carrier may be water or an aqueous based liquid carrier.

In other embodiments, the method comprises forming a solution or slurry of the solid organic dye or pigment in the water. In these embodiments, the other solvent is water and the liquid carrier may be an organic solvent, for example, methanol.

Note that in some embodiments, the method provides stable dispersions which do not contain a wetting agent or a dispersant at all or contain only a wetting agent or a dispersant. By contrast, a method relying upon dispersion of a milled solid organic dye or pigment generally requires both a wetting agent and dispersant.

In other embodiments, the method provides dispersions wherein the amount of the wetting agent and/or the amount of dispersant is substantially different to the amounts used to prepare similar dispersions following milling.

Note that the method does not require a solution or liquid carrier in its near critical or supercritical state. In one embodiment, however, the method uses a liquid carrier, for example water, in its near critical or supercritical state.

The method does not require that the density of the solution or slurry is different to that of the liquid carrier—but the organic or other solvent for the solution or slurry should be miscible with the liquid carrier.

The median (or Z) diameter size of the nanoparticles of the solid organic dye or pigment and the stability of the dispersions may be controlled by selection in one or more of the organic or other solvent and the liquid carrier and/or by selection in one or more process parameters.

These process parameters may include the concentration of the solution, the temperature and pressure of each of the solution or slurry and the liquid carrier, the residence times of the solution or slurry and the liquid carrier, and the ratio of the flow rates of the solution or slurry and the liquid carrier in the reactor.

The temperature at which the method is carried out may, for example, range between room temperature and 450° C. The pressure may, for example, range between 1 MPa and 25 MPa.

The residence times of the solution or slurry and the liquid carrier in the reactor may, for example, range between 1 second and 5 minutes and the ratio of flow rates may, for example, range between 1:1 and 1:100.

The selection may also control the polydispersity (mode and index) of the nanoparticle dispersion. In preferred embodiments, the method provides nanoparticle dispersions of the solid organic dye or pigment having unimodal polydispersity. The dynamic light scattering (DLS) polydispersity index of these and other dispersions may range between 0.1 and 3.0, and may, for example, be 2.0 or less, or 1.0 or less.

The organic pigment may be selected from those which are insoluble in a liquid carrier, such as water. Suitable pigments include alizarin, anthoxanthin, arylide yellow, azo dye, billin, bistre, caput mortuum, carmine, crimson, diarylide pigment, dibromoanthanthrone, dragon's blood, gamboge, indian yellow, indigo dye, naphthol AS, naphthol red, ommochrome, perinone, phthalocyanine blue BN, phthalocyanine green G, pigment blue 15:3, pigment violet 23, pigment yellow 10, pigment yellow 12, pigment yellow 13, pigment yellow 16, pigment yellow 81, pigment yellow 83, pigment yellow 139, pigment yellow 180, pigment yellow 185, pigment red 208, quinacridone, rose madder, rylene dye, sepia and tyrian purple.

In that case, the pigment is preferably, but not essentially, one which is soluble or sparingly soluble in the organic or other solvent at standard temperature and pressure.

The organic dye may be selected from those which are soluble in organic solvents but insoluble in a liquid carrier, such as water. Suitable dyes include, but are not limited to, disperse dyes such as Disperse Blue 14, Disperse Blue 19, Disperse Blue 72, Disperse Blue 334, Disperse Blue 359, Disperse Blue 360, Disperse Brown 27, Disperse Orange 25, Disperse Yellow 54, Disperse Yellow 64, Disperse Yellow 82, Disperse Red 55, Disperse Red 60, Macrolex Red H, Disperse Violet 28, Solvent Blue 67, Solvent Blue 70, Solvent Red 49, Solvent Red 160, Solvent Yellow 162, Solvent Violet 10, Solvent Black 29, Vat Red 41 and mixtures thereof.

The organic or other solvent may be a liquid or gas solvent. It may, in particular, comprise any suitable organic solvent including, but not limited to, ethyl acetate, ethanol, methanol, diethyl ether, tetrahydrofuran, dimethylformamide, dimethyl sulfoxide, N-methyl-2-pyrrolidone, acetone, isopropyl alcohol and mixtures thereof. It may alternatively comprise any suitable gas, and in particular, supercritical carbon dioxide.

The liquid carrier may be water or an aqueous based liquid carrier. The aqueous based liquid carrier may comprise water and one or more of a polyol, such as ethylene glycol, propylene glycol or a polyol having at least 5 carbon atoms, such as those described in International Patent Application WO 2014/127050 A1.

Alternatively, the other solvent may be water or an aqueous based liquid carrier as described above and the liquid carrier may be an organic solvent as described above.

The wetting agent and/or dispersant may comprise one or more water soluble surfactant. The water soluble surfactant may be an anionic surfactant or a non-ionic surfactant which is conventional to the manufacture of dye dispersions by milling.

Suitable anionic surfactants include, but are not limited to, alkyl sulfates, alkyl ether sulfates, alkyl aryl sulfonates (for example, a linear alkyl benzene sulfonate), α-olefin sulfonates, alkali metal or ammonium salts of alkyl sulfates, alkali metal or ammonium salts of alkyl ether sulfates, alkyl phosphates, silicone phosphates, alkyl glycerol sulfonates, alkyl sulfosuccinates, alkyl taurates, alkyl sarcosinates, acyl sarcosinates, sulfoacetates, alkyl phosphate esters, monoalkyl maleates, acyl isothionates, alkyl carboxylates, phosphate esters, sulfosuccinates, lignosulfonates and combinations thereof. Other suitable anionic surfactants include sodium lauryl sulfate, sodium lauryl ether sulfate, ammonium lauryl sulfosuccinate, ammonium lauryl sulfate, ammonium lauryl ether sulfate, sodium dodecylbenzene sulfate, triethanolamine dodecylbenzene sulfate, sodium cocoyl isothionate, sodium lauroyl isothionate and sodium N-lauryl sarcosinate.

Suitable non-ionic surfactants include, but are not limited to, mono- and di-alkanolamides, amine oxides, alkyl polyglucosides, ethoxylated silicones, ethoxylated alcohols, ethoxylated carboxylic acids, ethoxylated fatty acids, ethoxylated amines, ethoxylated amides, ethoxylated alkylolamides, ethoxylated alkylphenols, ethoxylated glyceryl esters, ethoxylated sorbitan esters, ethoxylated phosphate esters, block copolymers (for example, polyethylene glycol-polypropylene glycol block copolymers), glycol stearate, glyceryl stearate and combinations thereof.

The concentrating step of the method may be to remove only the organic or other solvent from the dispersion or may be to remove the organic or other solvent together with some of the liquid carrier.

The concentrating step of the method may be carried out by any suitable method—including evaporation, such as rotary evaporation under vacuum or a partial vacuum. In that case, the removal of the organic or other solvent may be carried out at a first temperature and the removal of liquid carrier may be carried out at a second temperature higher than the first.

The method may provide a nanoparticle dispersion of solid organic dye or pigment which can be directly used as an ink concentrate for inkjet printing.

In that case, the method requires concentrating the dispersion to remove at least the organic or other solvent and, optionally, a portion of the liquid carrier.

The method may provide a nanoparticle dispersion in which the solid content (viz. the concentration of the bare or encapsulated solid organic dye or pigment) is greater than 3 wt/wt % and less than or equal to 20 wt/wt %. In that case, the method may require concentrating the dispersion.

The solid content may, in particular, be from 5 wt/wt % to 20 wt/wt %, for example, from 5 wt/wt % to 15 wt/wt % or greater than 10 wt/wt % and less than 20 wt/wt % or 15 wt/wt %.

Further, the concentration of wetting agent in the dispersion may be from 0.5 wt/wt % to 5.0 wt/wt %, for example, 0.5 wt/wt % to 3.0 wt/wt %.

When present, the concentration of dispersant in the dispersion may be from 0.5 wt/wt % to 5.0 wt/wt %, for example 0.5 wt/wt % to 3.0 wt/wt %.

Of course, the concentrating step of the method may comprise complete centrifugation or filtration and resuspension of the solids in the liquid carrier.

In a second aspect, the present invention provides a method for obtaining nanoparticles of a solid organic dye or pigment, the method comprising forming a solution or slurry of the organic dye or pigment in an organic or other solvent and continuously mixing the solution or slurry with a liquid carrier in a counter current or concurrent mixing reactor providing a dispersion of nanoparticles of the solid organic dye or pigment in the liquid carrier and solvent mixture, and removing the nanoparticles of the solid organic dye or pigment from the dispersion.

Note that the precipitation or formation of the solid organic dye or pigment in the liquid carrier and solvent mixture may purify the solid organic dye or pigment in way which is similar to conventional re-crystallisation.

Accordingly, in a third aspect the present invention provides a method for the purification of a solid organic dye or pigment which method comprises the method according to the first or second aspect of the invention.

In a fourth aspect, the present invention provides a dispersion of nanoparticles of a solid organic dye or pigment obtained or obtainable from the method of the first aspect.

In a fifth aspect, the present invention provides a dispersion of nanoparticles of a solid organic dye or pigment in a liquid carrier, wherein the nanoparticles consist essentially of the solid organic dye or pigment.

In a sixth aspect, the present invention provides a dispersion of nanoparticles of a solid organic dye or pigment in a liquid carrier, wherein the nanoparticles consist essentially of the solid organic dye or pigment encapsulated, at least in part, by a wetting agent and/or dispersant.

In one embodiment, the dispersion may comprise a wetting agent and less than 5 wt/wt % of a dispersant.

In any case, the dispersion may have a solids content of the solid organic dye or pigment greater than 3 wt/wt % and less than 20 wt/wt %. The liquid carrier may be an aqueous based carrier. The dispersions may be stable at standard room temperature and pressure for longer than six months. The dispersions may be unimodal (as opposed to those obtained by milling). The dispersions may even comprise a solid organic dye or pigment (such as Disperse Red 55) that cannot be milled to a particle diameter below 500 nm.

In a seventh aspect, the present invention provides an ink concentrate for digital inkjet printing comprising the dispersion of the fifth or sixth aspect of the invention.

In an eighth aspect, the present invention provides a cosmetic paste comprising the dispersion of the fifth or sixth aspect of the invention.

Other aspects and embodiments will be apparent from the embodiments described in relation to the first aspect.

The present invention will now be described in more detail with reference to the following Examples and the accompanying drawings in which:

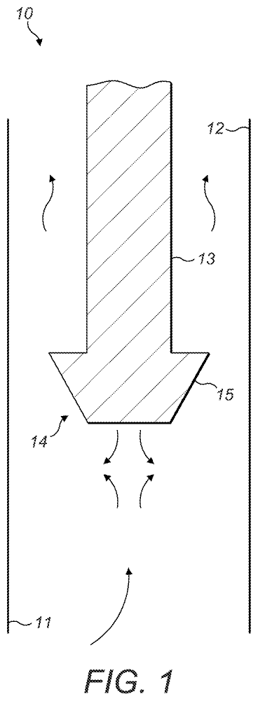

FIG. 1 is a schematic illustration of a counter current reactor, described in International Patent Application WO 2005/077505 A2, which is suitable for carrying out the method of the present invention;

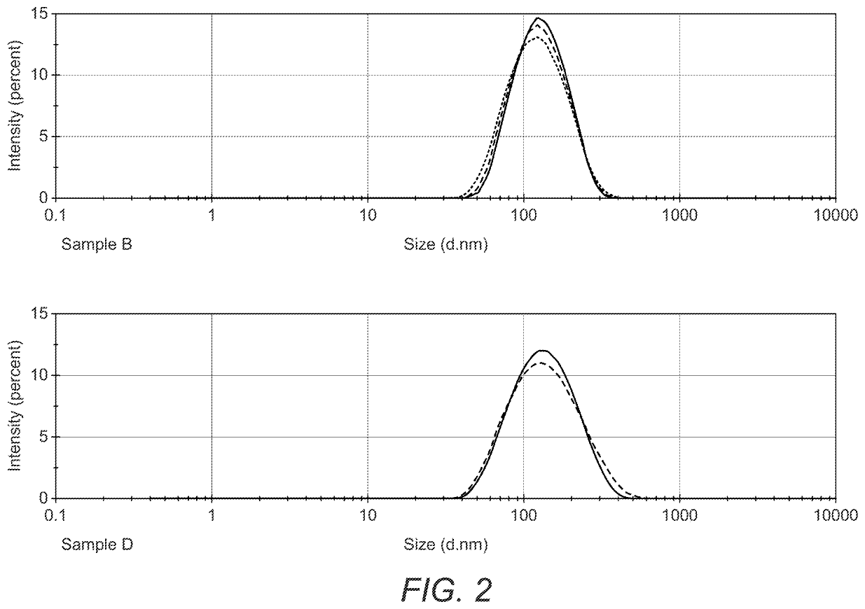

FIG. 2 is graph obtained by dynamic light scattering (DLS) from a dispersion prepared according to an embodiment of one method of the present invention;

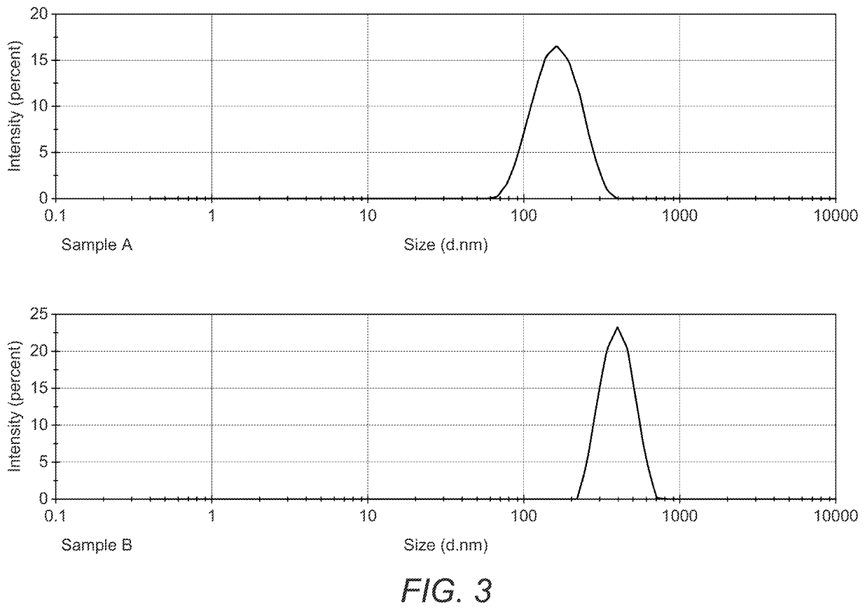

FIG. 3 is a graph obtained by DLS from a dispersion prepared according to another embodiment of the present invention; and

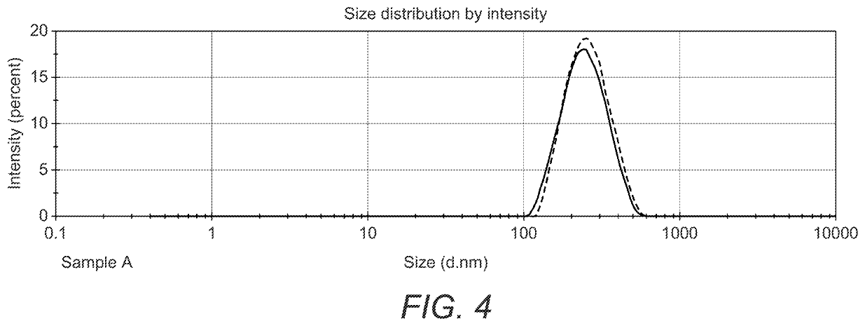

FIG. 4 is a graph obtained by DLS from a dispersion prepared according to still another embodiment of the present invention.

Referring now to FIG. 1, a counter current mixing reactor, generally designated 10, comprises a first inlet 11 and an outlet 12 in which a second inlet 13 is diametrically opposed to the first inlet 11 and disposed in the first inlet 11.

The first inlet 11 and the second inlet 13 are co-axial with one another and the second inlet 12 provides a nozzle 14 in the shape of a conical funnel 15.

A study of the preparation of several dispersions of nanoparticles of a solid organic dye (Disperse Red 60) in water was undertaken in a counter current mixing reactor as shown in FIG. 1 of laboratory scale.

The study examined the effect of variation in the ratio of flow rate of solutions of Disperse Red 60 in tetrahydrofuran (THF) with flow rate of water containing a surfactant (Morwet® D-425) as wetting agent/dispersant with the ratio of surfactant held constant.

In a first experiment, a downward flow of water was held at 20 ml/minute and an upward flow of THF solution was varied at values selected between 1 ml/minute and 20 ml/minute. The liquids were pumped by positive displacement pumps and the mixing performed at 25° C. and atmospheric pressure.

The concentration of the solution (g/L) of dye varied so as to keep the ratio of surfactant to dye constant at 8 whilst the ratio of flow rates varied.

The resulting dispersions were sampled (see Table 1, A to E) and the samples examined, after concentrating and decanting any sediment, by Dynamic Light Scattering (DLS).

TABLE 1
Variation of Flow Rates - Surfactant/Dye Ratio 8

| Sample | Water/THF | Surfactant/Dye | THF/Dye |
|---|---|---|---|
| A | 1.00 | 8.00 | 500 |
| B | 1.33 | 8.00 | 375 |
| C | 2.00 | 8.00 | 250 |
| D | 4.00 | 8.00 | 125 |
| E | 20.00 | 8.00 | 25 |

The samples were concentrated by rotary evaporation at room temperature (removing THF) followed by rotatory evaporation at 45° until a concentrate having a solid dye loading of about 10 to 15 wt/wt % was obtained.

The concentrated samples were prepared for examination by dilution of 1 mL of the supernatant fluid in 20 mL of deionised water. The diluted samples were analysed at 25° C. in a 10 mm cuvette using a Malvern Instruments Nano ZS particle sizer fitted with a back-scattering detector at 173° with an incident laser source (He—Ne laser with wavelength 632.8 nm).

A CONTIN algorithm was used to deconvolute the scattered light signal and give a size distribution. The analysis assumed a continuous phase of pure water (viscosity=0.8872 cP; refractive index=1.330) for the measurement settings. The Z-average size of the nanoparticles was taken from the raw cumulants data fit from the DLS instrument.

FIG. 2 shows the nanoparticle size distribution for two samples B and D of Table 1. As may be seen, the dispersions are unimodal and the Z-average (-median) particle size of the nanoparticles is respectively at 112 nm and 121 nm. The DLS polydispersity index of each sample was determined as 0.131 and 0.189 respectively.

In a second experiment, the downward flow of water was held at 20 ml/minute and the upward flow of THF solution varied between 1 ml/minute and 20 ml/minute with the ratio of surfactant to dye held constant at 24.

The liquids were pumped by positive displacement pumps and the mixing performed at 25° C. and atmospheric pressure.

The dispersion was sampled at different flow ratios (see Table 2, A to E) and the samples were concentrated and examined by Dynamic Light Scattering as described above.

TABLE 2
Variation of Flow Rates - Surfactant/Dye Ratio 24

| Sample | Water/THF | Surfactant/Dye | THF/Dye |
|---|---|---|---|
| A | 1.00 | 24.00 | 500 |
| B | 1.33 | 24.00 | 375 |
| C | 2.00 | 24.00 | 250 |
| D | 4.00 | 24.00 | 125 |
| E | 20.00 | 24.20 | 25 |

FIG. 3 shows the distribution of nanoparticle sizes for samples A and B of Table 2. As may be seen, the dispersions are unimodal and the Z-average (-median) particle size of the nanoparticles are respectively 170 nm and 396 nm. The DLS polydispersity index for each sample A and B was 0.280 and 0.267 respectively.

The effect of reaction temperature on the size of the nanoparticles was examined by repeating the second experiment in part A at a reaction temperature at 55° C. In this part, the flow ratio of water to THF was 1.00, the concentration of the dye in THF was 2 g/L and the concentration of surfactant in water was 48 g/L. The overall flow rate from the outlet of the reactor was 35 ml/min.

FIG. 4 shows the distribution of nanoparticle sizes for sample A at this temperature. As may be seen, the dispersion is unimodal and the Z-average (-median) particle size of the nanoparticles is 260 nm. The polydispersity index was 0.218.

Sample A did not show sedimentation although the other samples showed low but increasing sedimentation in line with decreasing THF content. Increasing the THF content in the mixing beyond that of the present study has been found to eliminate sedimentation and promote complete dispersion of the nanoparticles. All the samples in the study were stable.

Further studies indicate that stable dispersions of Disperse Red 60 can be obtained using acetone as the solvent without the need for the surfactant.

These studies taken together clearly show a method providing nanoparticle dispersions of a disperse dye and that the size and polydispersity index of the dispersions are sensitive to, and can be controlled by, the selection of parameters such as ratio of flow rate of the organic solvent with the liquid carrier. Other experiments also show that the method is also sensitive to the selection of organic solvent.

The present invention provides a single, continuous process for the preparation of stable dispersions of solid organic dye or pigment with desired nanoparticle size and encapsulation of the nanoparticles.

It also provides a single, continuous process for the purification of solid organic dye or pigment with desired nanoparticle size.

The present invention enables a large scale and environmentally responsible production of dye or pigment dispersion which generally avoids the large amounts of energy and solvent that are necessary for large scale milling.

The present invention may allow the preparation of nanoparticle dispersions or nanoparticles of organic dyes or pigments which cannot be milled effectively (for example, Disperse Red 55). It may, therefore, provide access to stable dispersions of solid organic dyes or pigments which are not presently obtainable. It may further provide access to new polymorphs of the crystalline organic dyes or pigments.

Note that the nanoparticle diameters specified herein are references to diameters which may be determined by, or calculated from, DLS of the dispersions in accordance with ISO 22412:2017. The solid contents specified herein are references to solid contents which may be determined by drying in accordance with ISO 3251:2008.

Note also that the nanoparticles of the present invention are not comprised by or reliant on an oil-in-water emulsion but are instead comprised by the solid organic dye or pigment or by the solid organic dye or pigment encapsulated (at least in part) by a water soluble surfactant.

Note further that the methods of the present invention may find general applicability to the preparation of nanoparticle dispersions and nanoparticles of other solid organic compounds—including pharmaceutical actives, pharmaceutical additives, pharmaceutical excipients, organometallic dopants or emitters useful in organic light emitting diodes (OLEDs) and organometallic catalysts useful in catalytic convertors and in organic synthesis.

1. A method for preparing a dispersion of nanoparticles of a solid organic dye or pigment in a liquid carrier, the method comprising i) forming a solution or slurry of the organic dye or pigment in an organic or other solvent, ii) continuously mixing the solution or slurry with the liquid carrier in a counter current or concurrent mixing reactor providing a dispersion of the nanoparticles in the liquid carrier and solvent mixture, and optionally, iii) concentrating the dispersion.

2. A method according to claim 1, wherein one or other of the solution or slurry and the carrier liquid contains a wetting agent and/or a dispersant.

3. A method according to claim 1, further comprising adding a wetting agent and/or a dispersant to the dispersion.

4. A method according to claim 1, wherein the method provides a dispersion of nanoparticles of median (Z) diameter between 100 nm and 300 nm, for example, between 100 nm and 150 nm.

5. A method according to claim 1, wherein the method provides a dispersion having unimodal polydispersity.

6. A method according to claim 1, wherein the method provides a solid content of the dispersion of the solid organic dye or pigment is greater than 5.0 wt/wt % and less than 15 wt/wt % of the dispersion.

7. A method according to claim 1, wherein the method provides a dispersion having a dynamic light scattering (DLS) polydispersity index between 0.1 and 3.0.

8. A method according to claim 1, comprising controlling one or more of nanoparticle size and polydispersity by selection in one or more of the organic or other solvent, the liquid carrier, the temperature and pressure of each of the solution or slurry and the liquid carrier, the residence times of the solution or slurry and the liquid carrier, and the ratio of the flow rates of the solution or slurry and the liquid carrier in the reactor.

9. A method according to claim 1, wherein the liquid carrier comprises water.

10. A method according to claim 9, wherein the organic solvent comprises one or more of ethyl acetate, ethanol, methanol, diethyl ether, tetrahydrofuran, dimethylformamide, dimethyl sulfoxide, N-methyl-2-pyrrolidone, acetone, ethylene glycol, propylene glycol and isopropyl alcohol.

11. A method according to claim 1, used for purifying the solid organic compound.

12. A dispersion of nanoparticles of a solid organic dye or pigment in a liquid carrier, obtained or obtainable by the method of claim 1.

13. A dispersion of nanoparticles of a solid organic dye or pigment in a liquid carrier, wherein the nanoparticles consist essentially of the solid organic dye or pigment.

14. A dispersion of nanoparticles of a solid organic dye or pigment in a liquid carrier, wherein the nanoparticles consist essentially of the solid organic dye or pigment and a wetting agent for the nanoparticles.

15. A dispersion according to claim 14, comprising less than 5 wt/wt % of a dispersant.

16. A dispersion according to claim 12, having a solid content of nanoparticles greater than 3 wt/wt % and less than 20 wt/wt %.

17. A dispersion according to claim 12, which has unimodal polydispersity.

18. A dispersion according to claim 12, wherein the nanoparticles have median (Z) diameter between 100 nm and 300 nm, for example, between 100 nm and 150 nm.

19. A dispersion according to claim 12, wherein the liquid carrier comprises water.

20. An ink concentrate for digital inkjet printing, comprising the dispersion of claim 12.

21. A cosmetic paste, comprising the dispersion of claim 12.
